(19) United States
(12) Patent Application Publication
(10) Pub. No.: US 2019/0148010 A1
(43) Pub. Date: May 16, 2019

(54) SYSTEM AND METHOD FOR CONTROLLING SENSING DEVICE

(71) Applicant: Samsung Electronics Co., Ltd., Suwon-si (KR)

(72) Inventor: Alireza Aliamiri, San Jose, CA (US)

(21) Appl. No.: 15/892,253

(22) Filed: Feb. 8, 2018

Related U.S. Application Data
(60) Provisional application No. 62/585,976, filed on Nov. 14, 2017.

(51) Int. Cl. G16H 40/67; A61B 5/024; A61B 5/00; G06F 1/16

(52) U.S. Cl. CPC G16H 40/67; G06F 1/163; A61B 5/0002; A61B 5/02438

(57) ABSTRACT

In a method for controlling a sensing device, the method includes: transmitting, by a processor, a signal to a wearable device to initiate vital signal sensing during a first time period; receiving, by the processor from the wearable device, vital signal data from the wearable device; adjusting, by the processor, a schedule for initiating the vital signal sensing based on the vital signal data; and transmitting, by the processor, a signal to the wearable device to initiate the vital signal sensing during a second time period according to the schedule for initiating the vital signal sensing.

### CROSS-REFERENCE TO RELATED APPLICATION(S)

This application claims priority to and the benefit of U.S. Provisional Patent Application No. 62/585,976 filed Nov. 14, 2017 and entitled “METHOD AND APPARATUS FOR SAVING POWER ON MEDICAL SENSING DEVICE”, the entire content of which is incorporated herein by reference.

### BACKGROUND

A wearable device (e.g., a watch, a bracelet) may have mounted thereon one or more sensors for detecting a vital signal anomaly or monitoring relevant vital signal data (e.g., an abnormal heart rhythm) of a user wearing the wearable device. Wearable devices may be powered using a battery to enable users to avoid connecting the devices to an external power source, but depending on the operation of the wearable device, the battery life may be relatively short and require frequent recharging.

The above information disclosed in this Background section is only for enhancement of understanding of the background of the present disclosure, and therefore, it may contain information that does not form prior art.

### SUMMARY

One or more example embodiments of the present disclosure are directed to a system and method for controlling a sensing device.

According to one or more embodiments, in a method for controlling a sensing device, the method includes: transmitting, by a processor, a signal to a wearable device to initiate vital signal sensing during a first time period; receiving, by the processor from the wearable device, vital signal data from the wearable device; adjusting, by the processor, a schedule for initiating the vital signal sensing based on the vital signal data; and transmitting, by the processor, a signal to the wearable device to initiate the vital signal sensing during a second time period according to the schedule for initiating the vital signal sensing.

According to some embodiments, the method further includes receiving, by the processor from the wearable device, contextual data corresponding to the vital signal data.

According to some embodiments, the contextual data comprises a time of the vital signal sensing.

According to some embodiments, the contextual data comprises motion information corresponding to the wearable device.

According to some embodiments, the method further includes determining, by the processor, whether or not to initiate a sensing interval during a second time period based on the vital signal data.

According to some embodiments, the method further includes calculating, by the processor, a probability of sensing a relevant vital signal during a portion of the second time period; and adjusting, by the processor, the schedule for initiating the vital signal sensing based on the calculated probability.

According to some embodiments, the signal to the wearable device to initiate the vital signal sensing during the first time period comprises a first instruction to perform the vital signal sensing during a first plurality of sensing intervals according to the schedule for initiating the vital signal sensing, and the signal to the wearable device to initiate the vital signal sensing during the second time period comprises a second instruction to perform the vital signal sensing during a second plurality of sensing intervals according to the schedule after the schedule is adjusted.

According to some embodiments, a duration and an interval of each of the first and second plurality of sensing intervals are defined by the schedule for initiating the vital signal sensing.

According one or more example embodiments, in a system for controlling a sensing device, the system includes: a processor; and a memory coupled to the processor, wherein the memory stores instructions that, when executed by the processor, cause the processor to: transmit a signal to a wearable device to initiate vital signal sensing during a first time period; receive, from the wearable device, vital signal data from the wearable device; adjust a schedule for initiating the vital signal sensing based on the vital signal data; and transmit a signal to the wearable device to initiate the vital signal sensing during a second time period according to the schedule for initiating the vital signal sensing.

According to some embodiments, the instructions further cause the processor to receive, from the wearable device, contextual data corresponding to the vital signal data.

According to some embodiments, the contextual data comprises a time of the vital signal sensing.

According to some embodiments, the contextual data comprises motion information corresponding to the wearable device.

According to some embodiments, the instructions further cause the processor to determine whether or not to initiate a sensing interval during a second time period based on the vital signal data.

According to some embodiments, the instructions further cause the processor to: calculate a probability of sensing a relevant vital signal during a portion of the second time period; and adjust the schedule for initiating the vital signal sensing based on the calculated probability.

According to some embodiments, the signal to the wearable device to initiate the vital signal sensing during the first time period comprises a first instruction to perform the vital signal sensing during a first plurality of sensing intervals according to the schedule for initiating the vital signal sensing, and the signal to the wearable device to initiate the vital signal sensing during the second time period comprises a second instruction to perform the vital signal sensing during a second plurality of sensing intervals according to the schedule after the schedule is adjusted.

According to some embodiments, a duration and an interval of each of the first and second plurality of sensing intervals are defined by the schedule for initiating the vital signal sensing.

According to one or more example embodiments, a sensor system includes: a server configured to schedule and initiate, according to the schedule, sensing intervals during a first time period for a wearable device located remotely with respect to the server; and the wearable device comprising one or more sensors for sensing vital signals of a user, wherein the wearable device is configured to sense location data and vital signal data and transmit the sensed location data and vital signal data to the server, wherein the wearable device is configured to activate sensors according to a signal from the server, and wherein the server is configured to adjust the schedule for a second time period for activating the sensors according to the sensed location data and vital signal data.

According to some embodiments, the server is configured to calculate a probability of sensing a relevant vital signal during a portion of the second time period and adjust the schedule for the second time period based on the calculated probability.

According to some embodiments, the server is further configured to receive, from the wearable device, contextual data corresponding to the vital signal data.

According to some embodiments, the server is further configured to determine whether or not the vital signal data comprises information indicating a vital signal anomaly based on the contextual data.

### BRIEF DESCRIPTION OF THE DRAWINGS

A more complete appreciation of the present disclosure, and many of the attendant features and aspects thereof, will become more readily apparent as the disclosure becomes better understood by reference to the following detailed description when considered in conjunction with the accompanying drawings in which like reference symbols indicate like components, wherein:

FIG. 1 illustrates an example vital signal detection system, according to one embodiment;

FIG. 2 illustrates an example diagram of an active learning process implemented by the vital signal detection system, according to one embodiment;

FIG. 3 is a diagram illustrating a frequency of activating sensors of a wearable medical device, according to one embodiment;

FIG. 4 is a timing diagram illustrating an example process of training a vital signal detection system, according to one embodiment;

FIG. 5 is a flow diagram illustrating a process of controlling a vital signal detection system, according to one embodiment;

FIG. 6A is a block diagram of a computing device according to according to one embodiment;

FIG. 6B is a block diagram of a computing device according to according to one embodiment;

FIG. 6C is a block diagram of a computing device according to one embodiment;

FIG. 6D is a block diagram of a computing device according to one embodiment; and

FIG. 6E is a block diagram of a network environment including several computing devices according to one embodiment.

### DETAILED DESCRIPTION

Hereinafter, example embodiments will be described in more detail with reference to the accompanying drawings, in which like reference numbers refer to like elements throughout. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of” and “at least one selected from,” when preceding a list of elements, modify the entire list of elements and do not modify the individual elements of the list.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively.

The electronic or electric devices and/or any other relevant devices or components according to embodiments of the present disclosure described herein, such as a processor, neural networks, neural network based controllers, a motor, actuators, and various sensors may be implemented utilizing any suitable hardware, firmware (e.g. an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the exemplary embodiments of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Wearable medical devices are gaining traction and enabling diagnosis and monitoring of patients outside of hospitals or medical facilities. Wearable medical devices enable users and their doctors to monitor vital signals, such as heart rate, blood oxygen levels, and a variety of other vital signals, and detect anomalies in such vital signals, using sensors included within the wearable medical device. The progression of modern technology has enabled wearable medical devices, and their components (e.g., sensors and sophisticated computing and communication components) to be small enough that users can wear such devices on their bodies as they go about their day, without the need to be tethered or hard-wired to an external power source or computing hardware.

In order to improve the user experience, including users' mobility during use, the components of wearable medical devices may be powered by an internal battery, instead of the wearable medical device being connected at all times to an external power source. The process of collecting vital signals and processing the corresponding data in order to facilitate a medical diagnosis or monitor a medical condition results in the stored of the battery to dissipate over time. The sensors of a wearable medical device that operate continuously or at frequent intervals may result in collection of significant instances of extraneous or irrelevant data that is not of interest for the purposes of monitoring a particular medical condition or symptom. Such extraneous collection of data may result in a relatively reduced battery life, and lead to relatively more frequent recharging of the battery of the wearable medical device. The reduced efficiency of battery consumption and reduced operating time from each charge of the battery from the inefficient collection of extraneous and irrelevant data may also result in reduced efficacy of the wearable medical device.

Thus, some example embodiments include a vital signal anomaly detection system and method that utilizes an active learning methodology that works with a wearable medical device to “wake up” or turn on the sensors of the wearable medical device to collect vital signal data (e.g., a heart rate/rhythm, a blood oxygen level, a blood pressure level, a body temperature, and a respiration rate) more frequently (or only) during time periods in which interesting or relevant vital signal anomaly data is generated by the user, and less frequently (or never) during time periods in which the user is not generating interesting or relevant vital signal data.

Relevant vital signal anomaly data may include, for example, detection of a vital signal that has a feature, a characteristic, or a value that is outside a predetermined threshold range or level of what would be expected, healthy, or normal for the user. For example, in the context of heart rate, a heart rate that exceeds a predetermined threshold level may be considered to constitute a vital signal anomaly. Similarly, in the context of a heart rhythm, a heart rhythm that is abnormal or irregular may be considered to constitute a vital signal anomaly. In some embodiments, the system may view the vital signal data in the context of other environmental or operating conditions of the wearable device. For example, if the vital signal sensing device is in motion, the vital signal anomaly detection system may determine that the user is engaged in physical activity to justify an elevated heart rate such that a detected elevated heart rate may not be considered to constitute a vital signal anomaly. Embodiments of the present disclosure are not limited to the above-described factors or mechanisms for determining whether or not a detected vital signal constitutes relevant vital signal anomaly data, and may include any other suitable factors, characteristics, or metrics for determining whether or not a detected vital signal is anomalous (e.g., outside the boundaries of normal, healthy, or acceptable ranges or levels). For example, further details of a system for determining that a detected vital signal constitutes a vital signal anomaly is described in U.S. Patent Application No. 62/581,569, entitled “Method and Apparatus for High Accuracy Photoplethysmogram Based Atrial Fibrillation Detection Using Wearable Device”, filed on Nov. 3, 2017, the disclosure of which is included in the Appendix filed herewith, and the entirety of which is incorporated by reference herein.

By controlling the time periods in which vital signal data is sensed and collected, some example embodiments may operate to maximize collection of relevant vital signal data, and minimize collection of irrelevant or uninteresting vital signal data, while also increasing or maximizing the operation time of the wearable medical device for each charge of the device's battery. Thus, embodiments of the present disclosure may enable controlling the activation of sensors in a wearable sensing device such that power consumption is reduced or minimized while preserving detection accuracy for vital signal anomalies.

According to some embodiments, the active learning methodology and controlling of the data collection by the wearable medical device may be executed on a cloud-based neural network machine learning system that is located remotely with respect to the user and the wearable medical device (although embodiments are not limited thereto, and such active learning and controlling may be executed internally by the wearable medical device according to other embodiments). As will be described in more detail below, a control and learning system may be in electronic communication with the wearable medical device (e.g., over a wireless communication channel) to receive vital signal data from the wearable medical device and to provide signals to the wearable medical device for activating and/or deactivating the sensing and collection of vital signal data. Because the active learning and controlling of data collection may occur on a remote cloud-based system, the vital signal detection system may further improve the wearable medical device's efficiency of battery charge consumption, because the amount of computing and processing executed by the wearable medical device may be reduced.

The present disclosure describes a cloud-based system and active learning methodology that works with a wearable device to wake up the wearable at times that of are interest without impacting the performance of sensing processes, that results in substantial power saving of the wearable sensor. The information that is used to determine the optimal time of sensing are collected from several sources including user location, a time of day, and a previous recorded relevant sensor data.

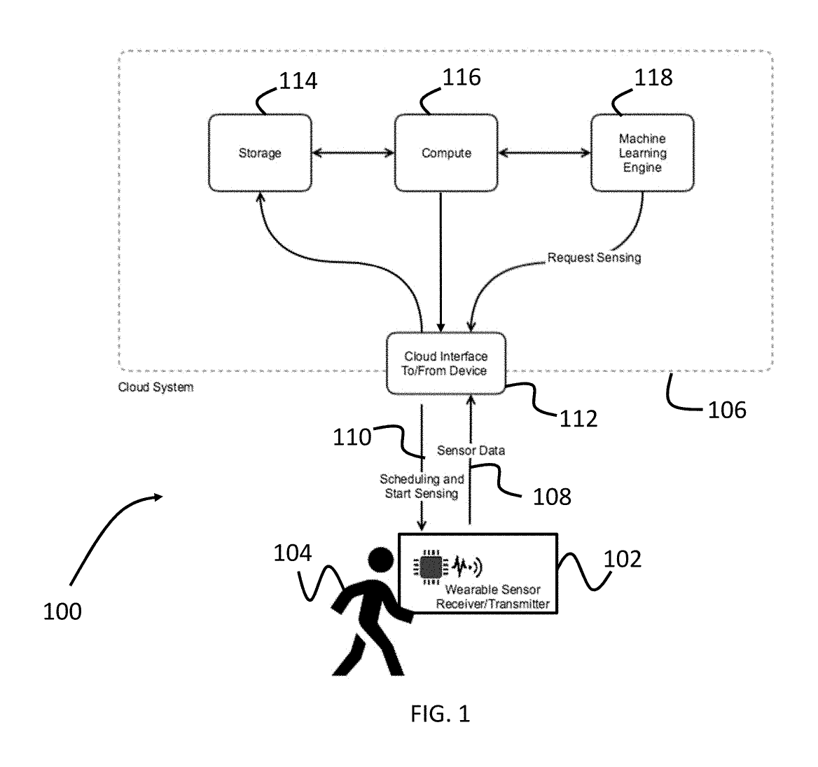

FIG. 1 illustrates an example vital signal detection system, according to some example embodiments. As shown in FIG. 1, a vital signal detection system 100 includes a wearable medical device 102 operated and/or worn by a user 104. As will be described in more detail below, the wearable medical device 102 may include one or more vital signal sensors configured to sense or detect vital signal data from the user 104 for facilitating monitoring or diagnosis of one or more medical conditions. For example, the vital signal sensors may include one or more photoplethysmogram (PPG) sensors, pulse oximeter, pulse wave velocity sensor, and any other suitable sensor configured to sense and/or collect vital signal data from a human body. Additionally, the wearable medical device 102 may include one or more operation sensors for measuring environmental or operational conditions of the wearable medical device, such as an inertial measurement unit (IMU), accelerometer, gyroscope, thermometer, clock, and/or any other suitable sensor for measuring relevant environmental or operational conditions of the wearable medical device. According to some embodiments, the wearable medical device 102 may further include electronic communication hardware (e.g., a receiver and/or transmitter) for communicating with external components.

The vital signal detection system 100 further includes a control system or server 106 in electronic communication with the wearable medical device 102, for example, by way of a wireless network configuration. The control system 106 is configured to receive data (e.g., sensor data and/or contextual/operational data) 108 from the wearable medical device 102 and transmit instructions 110 to the wearable medical device 102 to initiate and/or stop sensing and collecting vital signal data. According to some embodiments, the control system 106 may be in electronic communication with the wearable medical device 102 over a wireless data communication network (such as the Internet), and may include an interface module 112 for enabling the control system 106 and the wearable medical device 102 to exchange data and control signals (e.g., by way of an application programming interface (API)).

The control system 106 may include one or more storage or memory devices 114 configured to receive and store the data 108 received from the wearable medical device 102. The one or more memory devices 114 may further be connected to a processor, and may store instructions that, when executed by the processor, cause the processor to execute one or more operations for controlling and monitoring components of the vital signal detection system 100.

The control system 106 may further include a processor or computation module 116 configured to interface between the memory 114 and a machine learning engine or module 118, and to control one or more operations of the control system 106. For example, according to some embodiments, the computation module 116 may be configured to retrieve data stored in the memory 114 (e.g., sensor data 108) and calculate or determine whether or not the data includes information that is relevant to a vital signal or medical condition being monitored. The computation module 116 may then transmit the data (and/or the calculation or determination about the data) to the machine learning engine 118.

The machine learning engine 118 may have any suitable neural network architecture known to those skilled in the art, and may have been trained using any suitable sample data known to those skilled in the art. As will be discussed in more detail below, the machine learning engine 118 is configured to receive sensor data (or information about the sensor data) from the wearable medical device 102 (e.g., by way of the control system 106, the interface module 112, the one or more memory devices 114, and/or the computation module 116). The sensor data may include vital signal data as well as contextual information, such as motion, inertia, movement, environmental, and/or time data. In response to, receiving the sensor data, the machine learning engine 118 may enable the vital signal detection system 100 to modify or adjust the schedule or frequency for which the sensors of the wearable medical device 102 are active or turned on. According to some embodiments, during an initial phase (e.g., a training period and/or when the wearable medical device 102 is initially utilized by the user 104), the vital signal detection system 100 (by way of the computation module 116 and/or the machine learning engine 118 and the interface module 112) may transmit an instruction to the wearable medical device 102 to activate or turn on sensing of data (e.g., vital signal and contextual data) during regular and/or uniform intervals. In certain sensing periods, the vital signal detection system 100 may determine that a vital signal anomaly or relevant vital signal data is detected, and in other sensing periods, the vital signal detection system 100 may determine that a vital signal anomaly or relevant vital signal data is not detected.

Over time, using the machine learning engine 118, the vital signal detection system 100 may adjust the frequency and/or duration of sensing periods with a goal of maximizing or increasing instances where a vital signal anomaly or relevant vital signal data is detected during a sensing period, and minimizing or decreasing instances where a vital signal anomaly or relevant vital signal data is not detected during a sensing period. Thus, the vital signal detection system 100 may be configured to adjust the frequency and/or duration of sensing periods such that the sensors are activated or are turned on when there is a relatively high (e.g., exceeding a predetermined threshold) probability of detecting a vital signal anomaly or relevant vital signal data, and the sensors are not activated or are turned off when there is a relatively low (e.g., below a predetermined threshold) probability of detecting a vital signal anomaly or relevant vital signal data. Accordingly, the vital signal detection system 100 may be configured to conserve battery life and reduce recharging frequency by training itself to sense and collect data (thereby increasing battery charge consumption) when there is a high (e.g., exceeding a predetermined threshold) probability of sensing relevant data, and not sense and collect data (thereby reducing battery charge consumption) when there is a low (e.g., below a predetermined threshold) probability of sensing relevant data.

Error! Reference source not found. Illustrates an example diagram of an active learning process implemented by the vital signal detection system, according to some example embodiments. According to some embodiments, the vital signal detection system 100 may utilize any suitable neural network and/or deep learning architecture (such as a Deep Q Learning (DQN) architecture) to receive vital signal data as well as operational and/or contextual data from the wearable medical device 102, and adjust or control, by way of the machine learning engine 118, the frequency and/or duration for which the sensors of the wearable medical device 102 are activated.

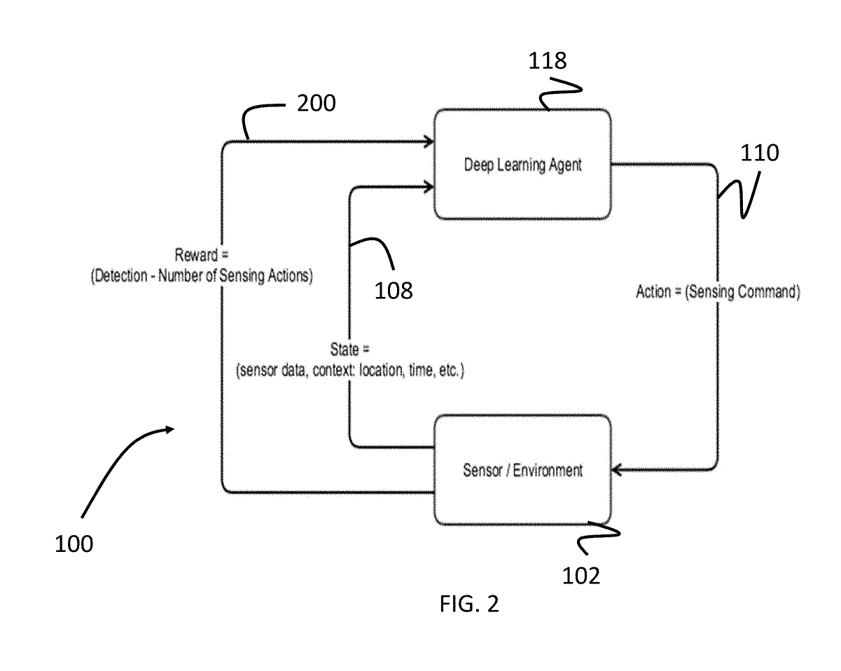

As illustrated in FIG. 2, the state s is transmitted to the machine learning engine 118 by way of the sensor data 108 from the wearable medical device 102. The state s includes sensor data, in which a vital signal anomaly or relevant vital signal may be detected, as well as various operational or contextual information, such as the location of the wearable medical device 102, the time the sensor data was collected or measured, the motion or inertia of the wearable medical device 102, various environmental condition measurements (e.g., temperature, humidity, etc.), whether or not one or more of the sensors is currently active, as well as previously measured or historical state information.

Additionally, because the vital signal detection system 100 may utilize a reinforcement DQN architecture, reward data 200 may be transmitted from the wearable medical device 102 to the machine learning engine 118. The reward, rₜ, of the reward data 200 is the goal to be maximized, which is having highest vital signal anomaly or relevant vital signal data detection rate while having lowest number of sensing attempts. The action, a, of the instructions 110 is a command to initiate a sensing operation from the one or more sensors of the wearable medical device 102.

The neural network of the machine learning engine 118 may be trained using simulated episodes of vital signal anomalies or relevant vital signal data generated from a physical model of the wearable medical device 102. An example goal of the neural network is to achieve best (or highest) vital signal anomaly or relevant vital signal data detection rate while having lowest number of sensing attempts.

The policy, π, is a mapping from state to actions, which tries to maximize or minimize a value function. The value function at each step represents how good each action or state is. A Q-value gives an expected total reward. A Q-value function gives the expected total reward from state s and action a under policy π with discount factor γ according to Equation [1], below:

Q^{π}(s, a)=E[rₜ₊₁+γrₜ₊₂+γ²rₜ₊₃+. . . |s, a] Equation [1]

An optimal value function is a maximum achievable value, which may be calculated according to Equation [2], below:

Q*(s, a)=max_{π}Q^{π}(s, a)=Q^{π}*(s, a) Equation [2]

The action to achieve the maximum achievable value may be calculated according to Equation [3], below:

π*(s)=argmaxₐQ^{π}*(s, a) Equation [3]

Thus, according to some embodiments of the present disclosure, a deep reinforcement learning model where a deep neural network (DQN) represents and learns the model, policy and value function may be utilized according to Equations [1]-[3] above. According to some example embodiments, a stochastic gradient descent may be utilized to optimize the loss function.

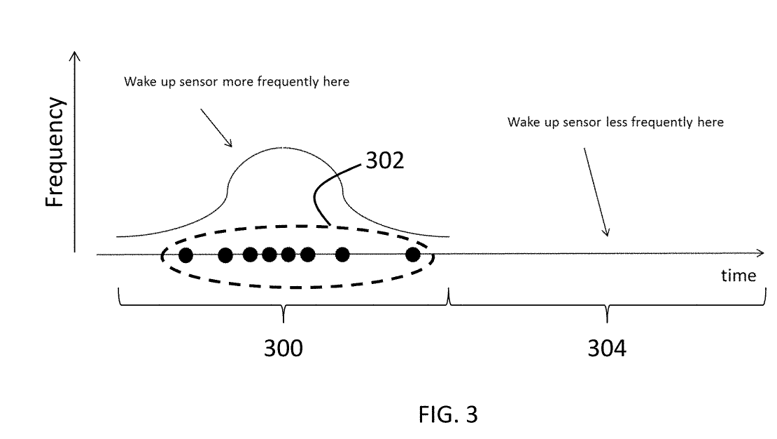

Error! Reference source not found. is a diagram illustrating a frequency of activating sensors of a wearable medical device, according to some example embodiments. As discussed above, the vital signal detection system 100 may be configured to adjust or modify the frequency for which the sensors of the wearable medical device 102 are activated, in order to increase or maximize the reward (e.g., detection of vital signal anomalies or relevant vital signal data) with the fewest number of sensing periods. Thus, as illustrated in FIG. 3, after training the vital signal detection system 100, the sensors of the wearable medical device 102 may be activated more frequently during a first period 300, in which there are a greater number of instances 302 of vital signal anomalies or relevant vital signal data being detected, compared to a second period 304, in which there are fewer instances (or no instances) of vital signal anomalies or relevant vital signal data being detected. The timing and duration of the first period 300 and the second period 304 may be determined based on the statistical probability of a vital signal anomaly or a relevant vital signal being detected, based on the determination of the machine learning engine 118.

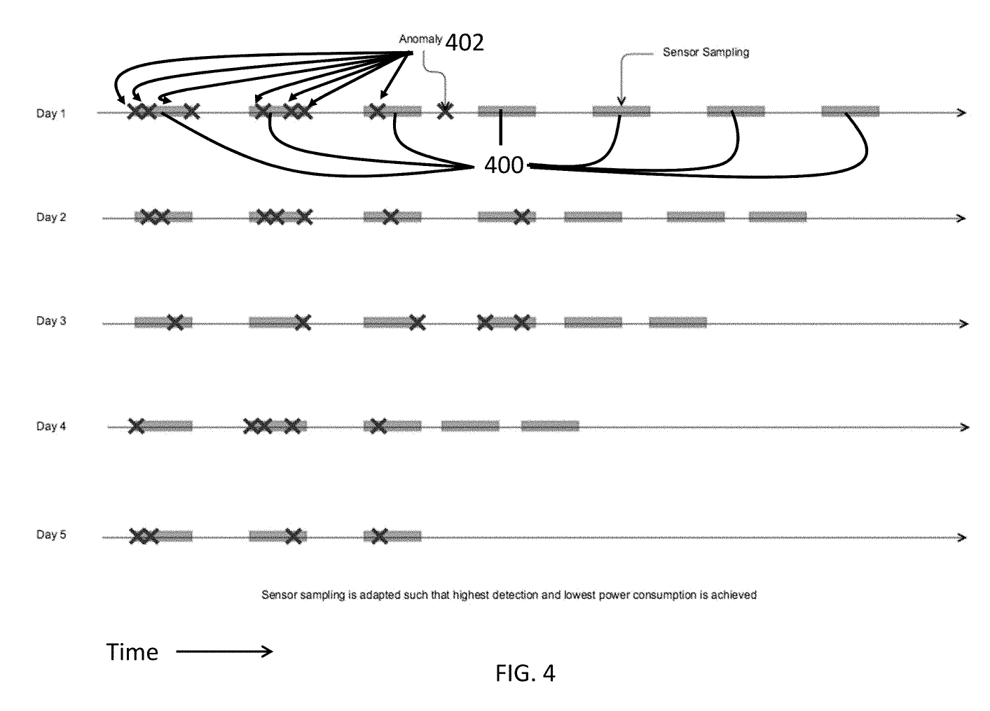

FIG. 4 is a timing diagram illustrating an example process of training a vital signal detection system, according to some example embodiments. Referring to FIG. 4, the vital signal detection system 100 may initiate sensing of vital signal data at periodic and/or uniform intervals 400 during a first training period (e.g., Day 1). During the intervals 400, the sensors of the wearable medical device 102 may be activated or turned on to senses or detect vital signal anomalies or relevant vital signal data. On the other hand, outside the intervals 400, the sensors of the wearable medical device 102 may be deactivated or turned off, such that the battery charge drawn by the wearable medical device 102 is reduced (compared to during the intervals 400), but the wearable medical device 102 cannot sense a vital signal anomaly or relevant vital signal data even if it occurs. During one or more of the sensing intervals 400, the vital signal detection system 100 may detect the occurrence of one or more instances of a vital signal anomaly or relevant vital signal data 402. Additionally, one or more instances of a vital signal anomaly or relevant vital signal data 402 may occur outside of any of the sensing intervals 400, such that they are not detected by the vital signal detection system 100. For each instance that a vital signal anomaly or relevant vital signal data 402 is detected or sensed during a sensing interval 400, the data 402 is transmitted to the control system 106, along with contextual and/or operational data (e.g., motion data, time of day, location information, environmental conditions, etc.). Additionally, according to some embodiments, contextual and/or operational data may be transmitted to the control system 106 for sensing intervals 400 during which no vital signal anomaly or relevant vital signal data 402 is detected.

Utilizing the data 402 and corresponding contextual and/or operational data, the vital signal detection system 100 may calculate a probability of detecting a vital signal anomaly or relevant vital signal data for various time periods using a suitable machine learning technique, such as explained above, and adjust (compared to the first training period) the frequency and/or duration of sensing intervals during a second training period (e.g., day 2) according to the calculated probabilities.

For each subsequent period (e.g., day 3, day 4, day 5, etc.), the vital signal detection system 100 continues to recalculate the probability of detecting a vital signal anomaly or relevant vital signal data for various time periods based on the data 402 and the corresponding contextual and/or operational data, and to readjust the frequency and/or duration of the sensing intervals based on the calculated probabilities, such that after multiple training periods (e.g., day 5) the vital signal detection system 100 initiates sensing intervals only during time periods in which there is a high probability (e.g., above a predetermined threshold probability) of detecting a vital signal anomaly or relevant vital signal data. During periods where there is a low probability (e.g., below a predetermined threshold probability) of detecting a vital signal anomaly or relevant vital signal data, the vital signal detection system 100 does not initiate a sensing interval.

According to some embodiments, the vital signal detection system 100 may not designate any particular number of training periods, but may update or adjust, on an ongoing or continuous basis, the frequency and/or duration of sensing intervals based on data 402 and corresponding contextual and/or operational data collected as part of previous sensing intervals.

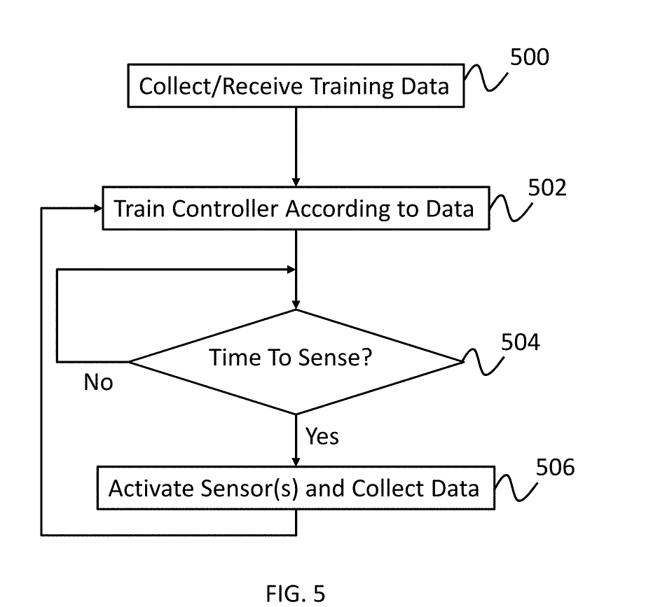

FIG. 5 is a flow diagram illustrating a process of controlling a vital signal detection system, according to some example embodiments. The number and order of operations in the process for controlling the vital signal detection system may vary according to various embodiments. That is, the process may include additional operations or fewer operations, and the relative order of the operations may vary unless otherwise stated expressly or implicitly. As illustrated in FIG. 5, at 500, the vital signal detection system 100 may collect and/or receive training data including state and contextual information, along with corresponding vital signal sensor data. At 502, the vital signal detection system 100 may train the vital signal detection controller, including the machine learning engine, to adjust the frequency and/or duration of sensing intervals based on the training data. According to some embodiments, the vital signal detection system 100 may not initially receive any training data, and instead may initiate sensing intervals according to a default or initial sensing interval schedule (e.g., consistent duration and evenly spaced intervals).

At 504, the vital signal detection system 100 determines whether or not it is time to initiate a sensing interval, based on a determination by the machine learning engine and/or a default sensing interval schedule, for activating or turning on sensors of the wearable medical device 102 to sense vital signals of the user. If, at 504, the vital signal detection system 100 determines it is not time to initiate a sensing interval, the vital signal detection system 100 cycles back to 504, to repeat the decision at another time (e.g., after a predetermined amount of time). If, at 504, the vital signal detection system 100 determines it is time to initiate the sensing interval, the vital signal detection system 100 transmits a signal to the wearable medical device 102 to activate or turn on one or more sensors of the wearable medical device 102 to initiate a sensing interval and begin collecting vital signal data of the user. At the end of the sensing interval, the vital signal detection system 100 may additionally transmit a signal to the wearable medical device 102 to deactivate or turn off the sensors that were turned on at the beginning of the sensing interval. Alternatively, the wearable medical device 102 may automatically deactivate the sensors after a predetermined period of time. After the sensing interval is completed, the vital signal detection system 100 transmits the vital signal sensor data and any corresponding contextual and/or operational data to the controller 106 to continue training the machine learning engine.

In one embodiment, each of the various servers, controllers, engines, and/or modules (collectively referred to as servers) in the afore-described figures are implemented via hardware or firmware (e.g. ASIC) as will be appreciated by a person of skill in the art.

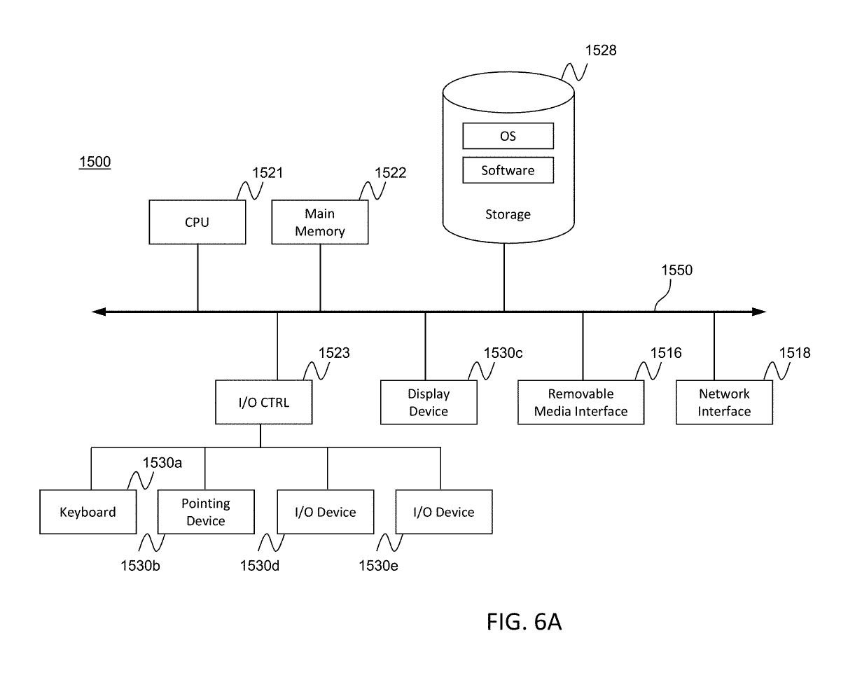

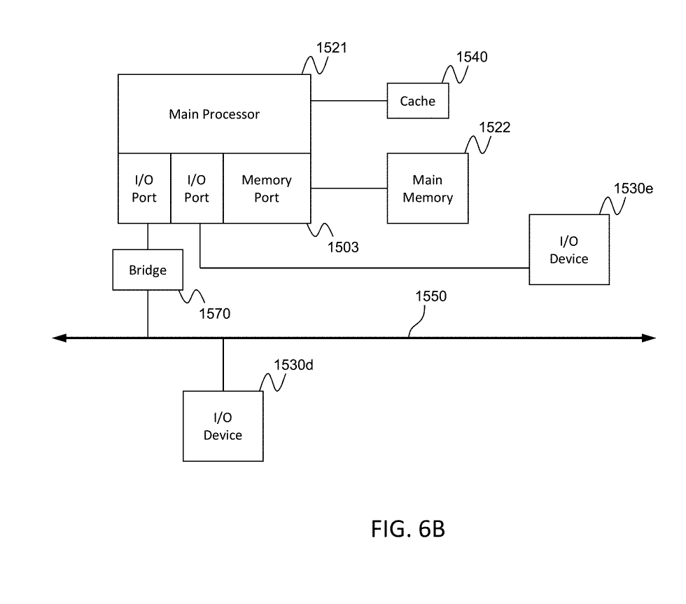

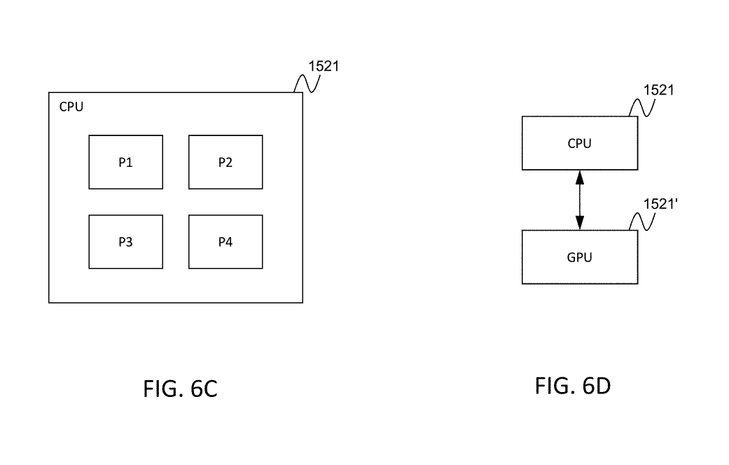

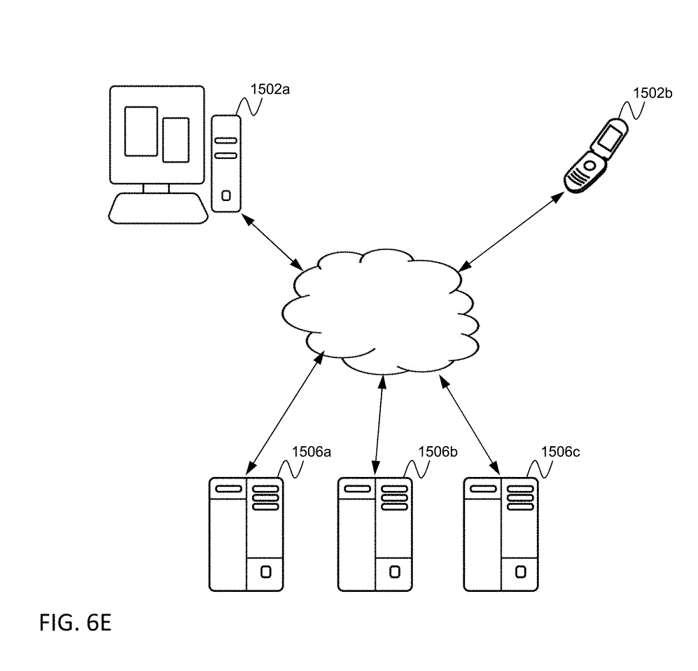

In one embodiment, each of the various servers, controllers, engines, and/or modules (collectively referred to as servers) in the afore-described figures may be a process or thread, running on one or more processors, in one or more computing devices 1500 (e.g., FIG. 6A, FIG. 6B), executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that a computing device may be implemented via firmware (e.g. an application-specific integrated circuit), hardware, or a combination of software, firmware, and hardware. A person of skill in the art should also recognize that, unless otherwise expressly stated or implied, the functionality of various computing devices may be combined or integrated into a single computing device, or the functionality of a particular computing device may be distributed across one or more other computing devices without departing from the scope of the example embodiments of the present disclosure. A server may be a software module, which may also simply be referred to as a module. The set of modules in the vital signal detection system may include servers, and other modules.

FIG. 6A and FIG. 6B depict block diagrams of a computing device 1500 as may be employed in the wearable medical device 102 and/or the control system 106 according to some example embodiments. Each computing device 1500 may include a central processing unit 1521 and a main memory unit 1522. As shown in FIG. 6A, the computing device 1500 may also include a storage device 1528, a removable media interface 1516, a network interface 1518, an input/output (I/O) controller 1523, one or more display devices 1530c, a keyboard 1530a and a pointing device 1530b, such as a mouse. The storage device 1528 may include, without limitation, storage for an operating system and software. As shown in FIG. 6B, each computing device 1500 may also include various additional optional elements, such as a memory port 1503, a bridge 1570, one or more additional input/output devices 1530d, 1530e and a cache memory 1540 in communication with the central processing unit 1521. The input/output devices 1530a, 1530b, 1530d, and 1530e may collectively be referred to herein using reference numeral 1530.

The central processing unit 1521 is any logic circuitry that responds to and processes instructions fetched from the main memory unit 1522. It may be implemented, for example, in an integrated circuit, in the form of a microprocessor, microcontroller, or graphics processing unit (GPU), or in a field-programmable gate array (FPGA) or application-specific integrated circuit (ASIC). The main memory unit 1522 may be one or more memory chips capable of storing data and allowing any storage location to be directly accessed by the central processing unit 1521. As shown in FIG. 6A, the central processing unit 1521 communicates with the main memory 1522 via a system bus 1550. As shown in FIG. 6B, the central processing unit 1521 may also communicate directly with the main memory 1522 via a memory port 1503.

FIG. 6B depicts an embodiment in which the central processing unit 1521 communicates directly with cache memory 1540 via a secondary bus, sometimes referred to as a backside bus. In other embodiments, the central processing unit 1521 communicates with the cache memory 1540 using the system bus 1550. The cache memory 1540 typically has a faster response time than main memory 1522. As shown in FIG. 6A, the central processing unit 1521 communicates with various I/O devices 1530 via the local system bus 1550. Various buses may be used as the local system bus 1550, including a Video Electronics Standards Association (VESA) Local bus (VLB), an Industry Standard Architecture (ISA) bus, an Extended Industry Standard Architecture (EISA) bus, a MicroChannel Architecture (MCA) bus, a Peripheral Component Interconnect (PCI) bus, a PCI Extended (PCI-X) bus, a PCI-Express bus, or a NuBus. For embodiments in which an I/O device is a display device 1530c, the central processing unit 1521 may communicate with the display device 1530c through an Advanced Graphics Port (AGP). FIG. 6B depicts an embodiment of a computer 1500 in which the central processing unit 1521 communicates directly with I/O device 1530e. FIG. 6B also depicts an embodiment in which local busses and direct communication are mixed: the central processing unit 1521 communicates with I/O device 1530d using a local system bus 1550 while communicating with I/O device 1530e directly.

A wide variety of I/O devices 1530 may be present in the computing device 1500. Input devices include one or more keyboards 1530a, mice, trackpads, trackballs, microphones, and drawing tablets. Output devices include video display devices 1530c, speakers, and printers. An I/O controller 1523, as shown in FIG. 6A, may control the I/O devices. The I/O controller may control one or more I/O devices such as a keyboard 1530a and a pointing device 1530b, e.g., a mouse or optical pen.

Referring again to FIG. 6A, the computing device 1500 may support one or more removable media interfaces 1516, such as a floppy disk drive, a CD-ROM drive, a DVD-ROM drive, tape drives of various formats, a USB port, a Secure Digital or COMPACT FLASH™ memory card port, or any other device suitable for reading data from read-only media, or for reading data from, or writing data to, read-write media. An I/O device 1530 may be a bridge between the system bus 1550 and a removable media interface 1516.

The removable media interface 1516 may for example be used for installing software and programs. The computing device 1500 may further comprise a storage device 1528, such as one or more hard disk drives or hard disk drive arrays, for storing an operating system and other related software, and for storing application software programs. Optionally, a removable media interface 1516 may also be used as the storage device. For example, the operating system and the software may be run from a bootable medium, for example, a bootable CD.

In some embodiments, the computing device 1500 may comprise or be connected to multiple display devices 1530c, which each may be of the same or different type and/or form. As such, any of the I/O devices 1530 and/or the I/O controller 1523 may comprise any type and/or form of suitable hardware, software, or combination of hardware and software to support, enable or provide for the connection to, and use of, multiple display devices 1530c by the computing device 1500. For example, the computing device 1500 may include any type and/or form of video adapter, video card, driver, and/or library to interface, communicate, connect or otherwise use the display devices 1530c. In one embodiment, a video adapter may comprise multiple connectors to interface to multiple display devices 1530c. In other embodiments, the computing device 1500 may include multiple video adapters, with each video adapter connected to one or more of the display devices 1530c. In some embodiments, any portion of the operating system of the computing device 1500 may be configured for using multiple display devices 1530c. In other embodiments, one or more of the display devices 1530c may be provided by one or more other computing devices, connected, for example, to the computing device 1500 via a network. These embodiments may include any type of software designed and constructed to use the display device of another computing device as a second display device 1530c for the computing device 1500. One of ordinary skill in the art will recognize and appreciate the various ways and embodiments that a computing device 1500 may be configured to have multiple display devices 1530c.

A computing device 1500 of the sort depicted in FIG. 6A and FIG. 6B may operate under the control of an operating system, which controls scheduling of tasks and access to system resources. The computing device 1500 may be running any operating system, any embedded operating system, any real-time operating system, any open source operating system, any proprietary operating system, any operating systems for mobile computing devices, or any other operating system capable of running on the computing device and performing the operations described herein.

The computing device 1500 may be any workstation, desktop computer, laptop or notebook computer, server machine, handheld computer, mobile telephone or other portable telecommunication device, media playing device, gaming system, mobile computing device, or any other type and/or form of computing, telecommunications or media device that is capable of communication and that has sufficient processor power and memory capacity to perform the operations described herein. In some embodiments, the computing device 1500 may have different processors, operating systems, and input devices consistent with the device.

In other embodiments the computing device 1500 is a mobile device, such as a Java-enabled cellular telephone or personal digital assistant (PDA), a smart phone, a digital audio player, or a portable media player. In some embodiments, the computing device 1500 comprises a combination of devices, such as a mobile phone combined with a digital audio player or portable media player.

As shown in FIG. 6C, the central processing unit 1521 may comprise multiple processors P1, P2, P3, P4, and may provide functionality for simultaneous execution of instructions or for simultaneous execution of one instruction on more than one piece of data. In some embodiments, the computing device 1500 may comprise a parallel processor with one or more cores. In one of these embodiments, the computing device 1500 is a shared memory parallel device, with multiple processors and/or multiple processor cores, accessing all available memory as a single global address space. In another of these embodiments, the computing device 1500 is a distributed memory parallel device with multiple processors each accessing local memory only. In still another of these embodiments, the computing device 1500 has both some memory which is shared and some memory which may only be accessed by particular processors or subsets of processors. In still even another of these embodiments, the central processing unit 1521 comprises a multicore microprocessor, which combines two or more independent processors into a single package, e.g., into a single integrated circuit (IC). In one exemplary embodiment, depicted in FIG. 6D, the computing device 1500 includes at least one central processing unit 1521 and at least one graphics processing unit 1521′.

In some embodiments, a central processing unit 1521 provides single instruction, multiple data (SIMD) functionality, e.g., execution of a single instruction simultaneously on multiple pieces of data. In other embodiments, several processors in the central processing unit 1521 may provide functionality for execution of multiple instructions simultaneously on multiple pieces of data (MIMD). In still other embodiments, the central processing unit 1521 may use any combination of SIMD and MIMD cores in a single device.

A computing device may be one of a plurality of machines connected by a network, or it may comprise a plurality of machines so connected. FIG. 6E shows an exemplary network environment. The network environment comprises one or more local machines 1502a, 1502b (also generally referred to as local machine(s) 1502, client(s) 1502, client node(s) 1502, client machine(s) 1502, client computer(s) 1502, client device(s) 1502, endpoint(s) 1502, or endpoint node(s) 1502) in communication with one or more remote machines 1506a, 1506b, 1506c (also generally referred to as server machine(s) 1506 or remote machine(s) 1506) via one or more networks 1504. In some embodiments, a local machine 1502 has the capacity to function as both a client node seeking access to resources provided by a server machine and as a server machine providing access to hosted resources for other clients 1502a, 1502b. Although only two clients 1502 and three server machines 1506 are illustrated in FIG. 6E, there may, in general, be an arbitrary number of each. The network 1504 may be a local-area network (LAN), e.g., a private network such as a company Intranet, a metropolitan area network (MAN), or a wide area network (WAN), such as the Internet, or another public network, or a combination thereof.

The computing device 1500 may include a network interface 1518 to interface to the network 1504 through a variety of connections including, but not limited to, standard telephone lines, local-area network (LAN), or wide area network (WAN) links, broadband connections, wireless connections, or a combination of any or all of the above. Connections may be established using a variety of communication protocols. In one embodiment, the computing device 1500 communicates with other computing devices 1500 via any type and/or form of gateway or tunneling protocol such as Secure Socket Layer (SSL) or Transport Layer Security (TLS). The network interface 1518 may comprise a built-in network adapter, such as a network interface card, suitable for interfacing the computing device 1500 to any type of network capable of communication and performing the operations described herein. An I/O device 1530 may be a bridge between the system bus 1550 and an external communication bus.

According to one embodiment, the network environment of FIG. 6E may be a virtual network environment where the various components of the network are virtualized. For example, the various machines 1502 may be virtual machines implemented as a software-based computer running on a physical machine. The virtual machines may share the same operating system. In other embodiments, different operating system may be run on each virtual machine instance. According to one embodiment, a “hypervisor” type of virtualization is implemented where multiple virtual machines run on the same host physical machine, each acting as if it has its own dedicated box. Of course, the virtual machines may also run on different host physical machines.

Other types of virtualization are also contemplated, such as, for example, the network (e.g. via Software Defined Networking (SDN)). Functions, such as functions of the session border controller and other types of functions, may also be virtualized, such as, for example, via Network Functions Virtualization (NFV).

Although the present disclosure has been described with reference to the example embodiments, those skilled in the art will recognize that various changes and modifications to the described embodiments may be performed, all without departing from the spirit and scope of the present disclosure. Furthermore, those skilled in the various arts will recognize that the present disclosure described herein will suggest solutions to other tasks and adaptations for other applications. It is the applicant's intention to cover by the claims herein, all such uses of the present disclosure, and those changes and modifications which could be made to the example embodiments of the present disclosure herein chosen for the purpose of disclosure, all without departing from the spirit and scope of the present disclosure. Thus, the example embodiments of the present disclosure should be considered in all respects as illustrative and not restrictive, with the spirit and scope of the present disclosure being indicated by the appended claims, and their equivalents. Further, those skilled in the art would appreciate that one or more features according to one more embodiments of the present disclosure may be combined with one or more other features according to one or more other embodiments of the present disclosure without departing from the spirit and scope of the present disclosure.

What is claimed is:

1. A method for controlling a sensing device, the method comprising:
transmitting, by a processor, a signal to a wearable device to initiate vital signal sensing during a first time period;
receiving, by the processor from the wearable device, vital signal data from the wearable device;
adjusting, by the processor, a schedule for initiating the vital signal sensing based on the vital signal data; and
transmitting, by the processor, a signal to the wearable device to initiate the vital signal sensing during a second time period according to the schedule for initiating the vital signal sensing.

2. The method of claim 1, further comprising receiving, by the processor from the wearable device, contextual data corresponding to the vital signal data.

3. The method of claim 2, wherein the contextual data comprises a time of the vital signal sensing.

4. The method of claim 2, wherein the contextual data comprises motion information corresponding to the wearable device.

5. The method of claim 1, further comprising determining, by the processor, whether or not to initiate a sensing interval during a second time period based on the vital signal data.

6. The method of claim 1, further comprising:
calculating, by the processor, a probability of sensing a relevant vital signal during a portion of the second time period; and
adjusting, by the processor, the schedule for initiating the vital signal sensing based on the calculated probability.

7. The method of claim 1, wherein the signal to the wearable device to initiate the vital signal sensing during the first time period comprises a first instruction to perform the vital signal sensing during a first plurality of sensing intervals according to the schedule for initiating the vital signal sensing, and
the signal to the wearable device to initiate the vital signal sensing during the second time period comprises a second instruction to perform the vital signal sensing during a second plurality of sensing intervals according to the schedule after the schedule is adjusted.

8. The method of claim 7, wherein a duration and an interval of each of the first and second plurality of sensing intervals are defined by the schedule for initiating the vital signal sensing.

9. A system for controlling a sensing device, the system comprising:
a processor; and
a memory coupled to the processor, wherein the memory stores instructions that, when executed by the processor, cause the processor to:
transmit a signal to a wearable device to initiate vital signal sensing during a first time period;
receive, from the wearable device, vital signal data from the wearable device;
adjust a schedule for initiating the vital signal sensing based on the vital signal data; and
transmit a signal to the wearable device to initiate the vital signal sensing during a second time period according to the schedule for initiating the vital signal sensing.

10. The system of claim 9, wherein the instructions further cause the processor to receive, from the wearable device, contextual data corresponding to the vital signal data.

11. The system of claim 10, wherein the contextual data comprises a time of the vital signal sensing.

12. The system of claim 10, wherein the contextual data comprises motion information corresponding to the wearable device.

13. The system of claim 9, wherein the instructions further cause the processor to determine whether or not to initiate a sensing interval during a second time period based on the vital signal data.

14. The system of claim 9, wherein the instructions further cause the processor to:
calculate a probability of sensing a relevant vital signal during a portion of the second time period; and
adjust the schedule for initiating the vital signal sensing based on the calculated probability.

15. The system of claim 9, wherein the signal to the wearable device to initiate the vital signal sensing during the first time period comprises a first instruction to perform the vital signal sensing during a first plurality of sensing intervals according to the schedule for initiating the vital signal sensing, and
the signal to the wearable device to initiate the vital signal sensing during the second time period comprises a second instruction to perform the vital signal sensing during a second plurality of sensing intervals according to the schedule after the schedule is adjusted.

16. The system of claim 15, wherein a duration and an interval of each of the first and second plurality of sensing intervals are defined by the schedule for initiating the vital signal sensing.

17. A sensor system comprising:
a server configured to schedule and initiate, according to the schedule, sensing intervals during a first time period for a wearable device located remotely with respect to the server; and
the wearable device comprising one or more sensors for sensing vital signals of a user,
wherein the wearable device is configured to sense location data and vital signal data and transmit the sensed location data and vital signal data to the server,
wherein the wearable device is configured to activate sensors according to a signal from the server, and
wherein the server is configured to adjust the schedule for a second time period for activating the sensors according to the sensed location data and vital signal data.

18. The sensor system of claim 17, wherein the server is configured to calculate a probability of sensing a relevant vital signal during a portion of the second time period and adjust the schedule for the second time period based on the calculated probability.

19. The sensor system of claim 17, wherein the server is further configured to receive, from the wearable device, contextual data corresponding to the vital signal data.

20. The sensor system of claim 19, wherein the server is further configured to determine whether or not the vital signal data comprises information indicating a vital signal anomaly based on the contextual data.
